(19) United States
(12) Patent Application Publication
(10) Pub. No.: US 2008/0195130 A1
(43) Pub. Date: Aug. 14, 2008

(54) Method and apparatus for catheter removal

(76) Inventor: Stanley Batiste, Las Vegas, NV (US)

Correspondence Address: WEIDE & MILLER, LTD., 7251 W. LAKE MEAD BLVD., SUITE 530, LAS VEGAS, NV, 89128, US

(21) Appl. No.: 12/069,885

(22) Filed: Feb. 12, 2008

Related U.S. Application Data
(60) Provisional application No. 60/901,294, filed on Feb. 12, 2007.

(51) Int. Cl. A61B 17/32

(52) U.S. Cl. 606/170

(57) ABSTRACT

A catheter removal device having a handle, a shaft extending from the handle, a cutting tip, and a longitudinal groove extending through the cutting tip and at least a portion of the shaft is disclosed. The cutting tip has a leading edge, a cutting edge, and delivery threads. The leading edge and the cutting edge are sharpened, and the delivery threads may be on the interior or the exterior of the cutting tip. A catheter is removed by attaching the device to an external portion of the catheter and advancing the device's cutting tip along the catheter. The leading edge and cutting edge allow the device to cut tissues attached to the catheter while the delivery threads help pull the device into the patient along the catheter. Once the connecting tissues or scar tissues are cut away the device and the catheter may be removed together.

### CROSS-REFERENCE TO RELATED APPLICATIONS

This application claims priority to U.S. Provisional Patent Application No. 60/901,294 filed on Feb. 12, 2007 titled METHOD AND APPARATUS FOR CATHETER REMOVAL.

### BACKGROUND OF THE INVENTION

1. Field of the Invention

The present invention relates to medical devices and, in particular to a surgical device for facilitating the removal of a vascular catheter.

2. Related Art

Permanent Dialysis Catheters are duel lumen relatively large bore catheters which are used for dialysis patients in need of large volume blood exchange. The catheter is generally used for hemodialysis and comprises two ports—one port to draw blood from the patient and a second port for blood return subsequent to dialysis. A permanent dialysis catheter is the only real long term catheter currently used which will “scar” in place. The dialysis catheter is placed under the skin to be less visible than other catheters and scars in place to prevent infection.

Permanent Dialysis Catheters are generally placed within the Internal Jugular vein (See FIG. 1). The main body of the catheter is “tunneled” under the patient's skin and exits from the upper chest (See FIG. 1). Tunneling is a method which places a large part of the catheter below the skin. It is performed by making and initial hole with an entry site in the vein. Several centimeters away from the vein entry site a second hole (dermatotomy) is made with a scalpel. Using a “tunneling device (stiff, blunt-ended rod) the catheter is advanced (tunneled) from the dermatotomy site to the initial puncture site at the vein beneath the patient's skin.

Along the tunneled section of the catheter there is a “bacteriostatic cuff” which is made to scar in place to prevent Bacteria from migrating from the skin to the deep tissues (See FIG. 1). The scarring essentially “seals” the tract in which the catheter is surgically implanted. The scarring adheres directly to the catheter making a barrier to infection, water or other undesirable substances. The scar tissue is very hard and difficult to cross or penetrate.

After several months of use, the permanent dialysis catheter needs to be replaced usually due to decreased functionality. Dialysis catheters have an average patency rate of approximately 80 days. The dialysis catheter typically becomes unusable due to fibrin (sheathing) deposits along the length of the catheter within the vein. The fibrin, which is a type of scarring in the veins, creates narrowing and eventual occlusion of the tips of the catheter. Fibrin typically covers the outside of the catheter and prevents the catheter from functioning properly. Another reason for catheter replacement is commonly infection. The infection may come from the entry point at the skin surface or from a blood-borne infection within the vein, in either way the catheter must be removed.

Since the dialysis catheter is designed to scar in place, it becomes very difficult to remove. The increased difficulty in removing the catheter is due to the large amount of scar tissue that builds up and adheres around the bacteriostatic cuff.

The current surgical method of removing a dialysis catheter is by using blunt dissection with a hemostat. The scarring adhered to the catheter is separated by advancing a hemostat into the tract region and along the catheter then spreading the hemostat to separate/tear the scar tissue planes. The hemostat is an “all purpose” medical tool that is less than optimal for catheter removal because the hemostat creates added trauma by tearing the tissues surrounding the catheter. As a result, the current method of dialysis catheter removal requires more time in order to perform the procedure. Additionally, due to the trauma to adjacent tissues, there is usually bleeding and a moderate amount of pain caused to the patient which may last for several days. The dialysis catheter removal as currently practiced has several disadvantages such as increased procedure time, localized tissue trauma, bleeding and associated complications, inducing patient pain and increased recovery time.

As a result, there is a need in the art for an improved method and apparatus that facilitates dialysis catheter removal which has the following characteristics: inexpensive, quickly performed with reduced tissue damage, reduces patient discomfort and minimal recovery time. The method and apparatus described herein enables a physician to efficiently remove a dialysis catheter with minimal physical intrusion and discomfort while at the same time reducing risk of procedural complications such as tissue trauma for the patient.

### SUMMARY OF THE INVENTION

To overcome the drawbacks of the prior art and provide additional benefits and features, a catheter removal device and method of catheter removal is disclosed. In one embodiment, the catheter removal device includes a handle, a shaft extending from the handle, and a cutting tip extending from the distal end of the shaft. The shaft may be semi-rigid in some embodiments, and includes a longitudinal groove which extends through the distal end of the shaft. The longitudinal groove is generally configured to accept the outer dimension of a catheter and thus may be substantially circular in some embodiments.

The cutting tip includes its own longitudinal groove, delivery threads, a leading edge having a honed cutting surface, and an internal cutting edge formed on the longitudinal groove of the cutting tip. The delivery threads may be formed on the longitudinal groove of the cutting tip or may be formed on an external surface of the cutting tip. The longitudinal groove of the cutting tip may also be configured to accept the outer dimension of a catheter and thus may also be substantially circular in some embodiments.

When removing a catheter, the catheter removal device is attached to an external portion of a catheter to be removed. In one embodiment, the device is attached by placing at least a portion of the longitudinal groove of the shaft, the cutting tip, or both around a portion of the catheter external to the patient. The device is then advanced along the catheter until the cutting tip engages a cuff portion of the catheter. Advancing is accomplished by applying a turning force and a forward force and may be assisted by the delivery threads and leading edge of one or more embodiments. Tissue may be excised or separated from the catheter, the catheter's cuff portion, or both by cutting away the connecting tissue such as by applying a turning force and a forward force to the device. In one or more embodiments, such force, causes the cutting edge, which extends into the longitudinal groove surrounding the catheter, to cut away the tissues surrounding the catheter. The catheter and the device may then be removed together upon separation of the tissue.

In some embodiments the cutting edge may be fixed, while in other embodiments the cutting edge may be retractable. For example, the cutting edge may not extend into the longitudinal grove of the cutting tip until cutting is necessary. When cutting is necessary, the cutting edge may be extended into the longitudinal groove to cut away tissues. In these embodiments, the cutting edge is deployed when it is so desired such as when tissue must be cut away from around the catheter, and the cutting edge is retracted when such cutting is not necessary.

In one or more embodiments, the cutting edge may be associated with an activation rod operatively coupled with a cutting switch and a longitudinal cavity in the catheter removal device's shaft. The longitudinal cavity allows the activation rod to longitudinally translate in response to movement of the cutting switch. The cutting switch is movable from a first position to a second position, and vice versa. Movement of the cutting switch causes corresponding motion of the activation rod which in turn deploys or retracts the cutting edge into the longitudinal groove of the cutting tip. In one embodiment, moving the cutting switch to a first position retracts the cutting edge while moving the cutting switch to the second position deploys the cutting edge. In some embodiments, the cutting edge may be integrally formed into the activation rod. Also, the embodiments with retractable cutting edges may include the various combinations of internal/external delivery threads, a semi-rigid shaft, and longitudinal grooves described above.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIG. 1 illustrates a typical dialysis catheter implant.

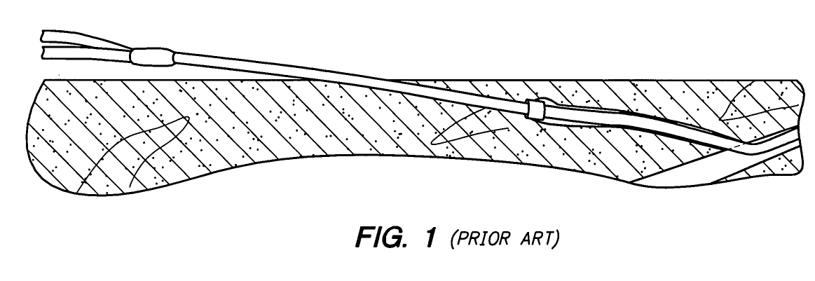

FIG. 2A illustrates one embodiment of a catheter removal device having internal delivery threads.

FIG. 2B illustrates another embodiment of the catheter removal device having external delivery threads.

FIGS. 3A and 3B illustrate another embodiment of a catheter removal device having a retractable cutting edge.

FIGS. 4 through 9 illustrate typical dialysis catheter implant removal utilizing the catheter removal device of the present invention.

### DETAILED DESCRIPTION OF THE PREFERRED EMBODIMENTS

In the following description, numerous specific details are set forth in order to provide a more thorough description of the present invention. It will be apparent, however, to one skilled in the art, that the present invention may be practiced without these specific details. In other instances, well-known features have not been described in detail so as not to obscure the invention.

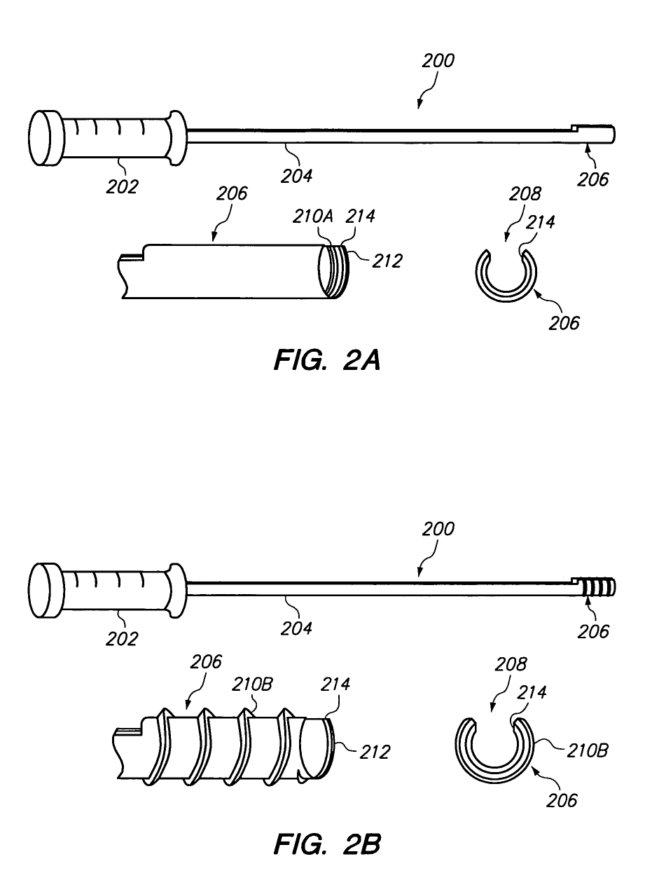

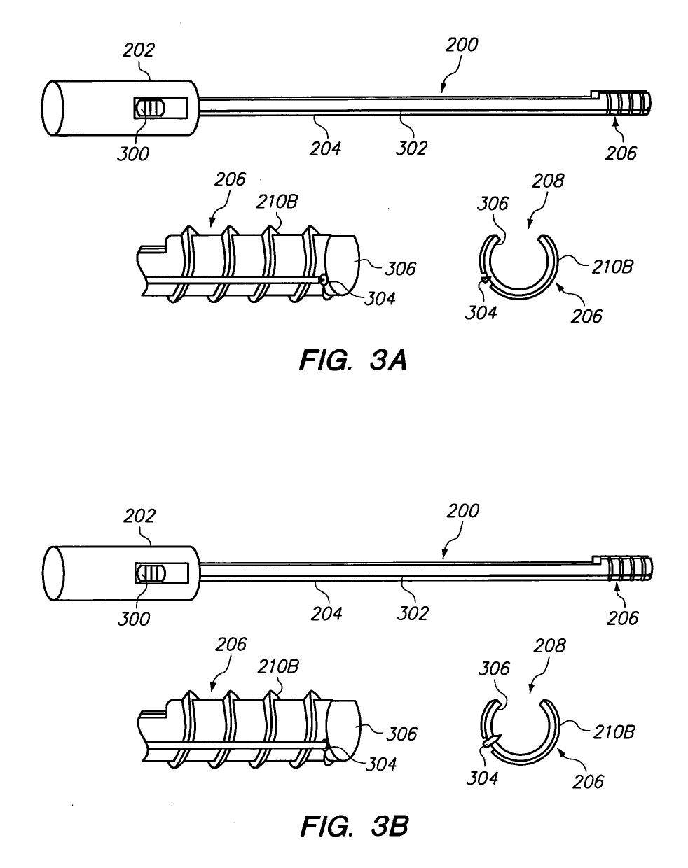

Referring now to the drawings, FIG. 2A illustrates one embodiment of a catheter removal device 200 which comprises a handle 202, a shaft 204 and a cutting tip 206. The handle 202 is contemplated to be sized and shaped for use in the human hand where the handle provides adequate griping surface to enable the user to twist and advance the catheter removal device 200. The handle may be fabricated using injection molding techniques from a suitable material such as high-strength plastic or other polymers/plastics. The shaft 204 is a substantially rigid member extending from the handle 202 and terminating at a distal end with an integrally formed cutting tip 206. The shaft 204 is generally hollow or configured with a “U-shaped” groove 208 axially extending from the handle 202 through the cutting tip 206. The “U-shaped” groove 208 is sized to accommodate the outer dimension of the catheter tubing by which the catheter removal device 200 may be attached to and advanced over the catheter tubing. The shaft 204 may be semi-rigid while at the same time providing adequate strength and stability to counter the torque and advancement forces that are applied during use. The shaft 204 and cutting tip 206 may be fabricated using injection molding/casting techniques from various materials such as high-strength polymers, plastics, metals, alloys or combinations thereof.

In FIG. 2A, the cutting tip 206 is illustrated in a side view and a frontal view. In the side view of FIG. 2A, the cutting tip 206 has a plurality of delivery threads 210A formed on the inner surface of the “U-shaped” groove 208. The delivery threads 210A are circumferentially formed ridges, rifling, ribs or lands that engage the outer surface of the catheter and as the tool is rotated, the delivery threads 210A draw the catheter removal device 200 along the length of the catheter and into the patient. It is contemplated that delivery threads 210A with various twist rates (turns per unit length) or thread pitches may be implemented as necessary to facilitate advancement. The tool is drawn into a patient along the catheter because the friction of the pitched or inclined delivery threads when rotated or turned causes the threads to pull or draw the tool further into the patient.

The cutting tip 206 has a circumferential leading edge 212 that is configured with a honed cutting surface. The leading edge 212 also facilitates advancement of the catheter removal device 200 by providing a leading edge cutting surface that cuts away tissue and thereby permits the device to advance into the patient. Additionally, the cutting tip 206 is configured with an internal circumferential cutting edge 214 that is oriented inward towards the center of the “U-shaped” groove 208. The cutting edge 214 is designed to separate scar tissue from the surface of the catheter and especially around the bacteriostatic cuff shoulder area.

As described below with reference to FIGS. 2B and 2C, some embodiments of the catheter removal device will have delivery threads. However, it is contemplated that not all embodiments will utilize delivery threads (external or internal) as the catheter removal device may be used to remove catheters without such threads such as described above.

An embodiment is shown in FIG. 2B, which illustrates a catheter removal device having external delivery threads 210B. In embodiments with delivery threads, the primary structure (e.g., handle, shaft, leading edge and cutting edge) of the catheter removal device 200 remains substantially similar to that previously described with reference to FIG. 2A. However, in this new embodiment, the delivery threads 210B are circumferentially formed on the exterior surface of the cutting tip 206. The external delivery threads 210B are circumferentially formed ridges, rifling, ribs or lands that engage the tissue surrounding the catheter and as the tool is rotated, the delivery threads 210B advance the catheter removal device 200 along the length of the catheter and into the patient. It is contemplated that delivery threads 210B with various twist rates (turns per unit length) or thread pitches may be implemented as necessary.

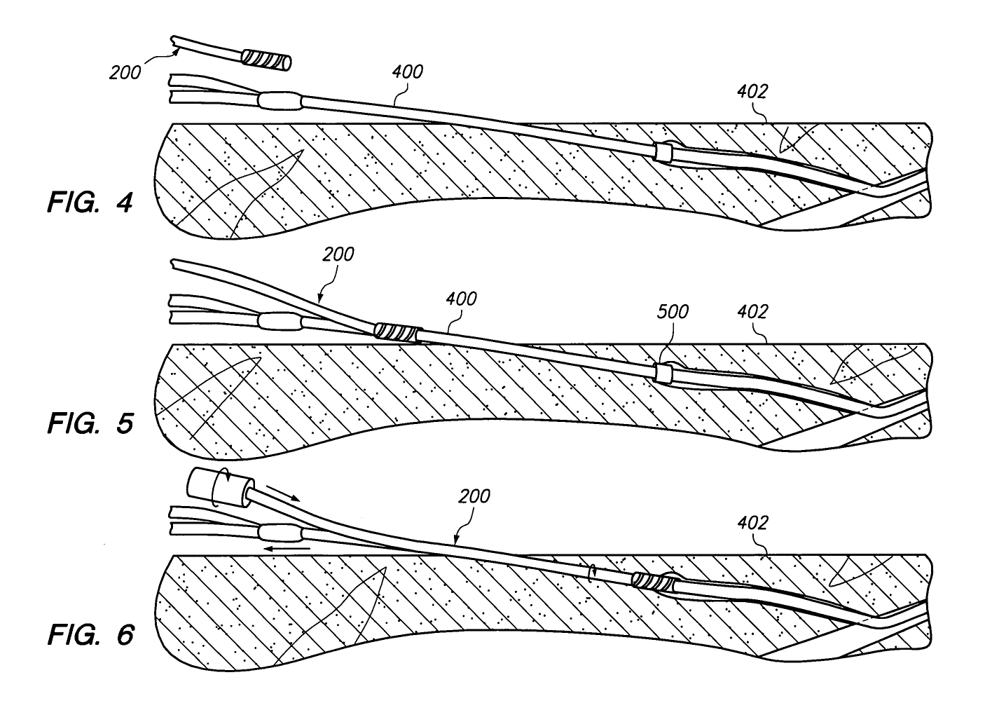

Another embodiment is shown in FIGS. 3A and 3B, which illustrate a catheter removal device having a retractable cutting edge. The primary structure (e.g., handle, shaft, delivery threads and leading edge) of the catheter removal device 200 remains substantially similar to that previously described with reference to FIGS. 2A and 2B. In FIG. 3A, a cutting switch 300 is illustrated in a retracted position. The cutting switch 300 is operatively linked with a longitudinal activation rod 302 which in turn is operatively coupled within the shaft 204. The activation rod 302 may longitudinally translate within a slot, cavity or chamber formed in the shaft 204. The activation rod 302 terminates at a distal end in which a retractable cutting edge 304 may be integrally formed thereon. Upon longitudinal translation of the rod 302, the rod forces or engages the retractable cutting edge 304 such that the cutting edge is deployed beyond the inner surface 306 of the cutting tip 206. The retractable cutting edge 304 is configured and orientated to extend inward towards the center of the “U-shaped” groove 208 upon activation of the cutting switch 300. The retractable cutting edge 304 is designed to separate scar tissue from the surface of the catheter and especially around the catheter cuff area.

In FIG. 3B, the cutting switch 300 is illustrated in the deployed position in which the activation rod 302 is advanced causing the retractable cutting edge 304 to extend into the “U-shaped” groove 208. Once the retractable cutting edge 304 is deployed into the groove 208 area, the catheter removal device 200 is then twisted, which in turn causes the cutting edge 304 to excise the scar tissue adhering to the catheter and cuff surfaces. After the scar tissue is separated from the catheter, the retractable cutting edge 304 may then be placed in the retracted position to facilitate removal of the device 200 or alternatively left deployed. It is further contemplated that the embodiment shown in FIGS. 3A and 3B may be modified to implement the internal delivery threads in lieu of the illustrated external delivery threads 210B or a combination of both internal and external delivery threads.

It is contemplated that other variations on the construction and configuration of the catheter removal device may be implemented. In one alternate configuration, the handle, shaft and cutting tip may be fabricated as a unitary construction such as a single piece injection molded device. In another variation, there may only be one excising surface such as only the leading edge. Additionally, another embodiment may be configured with a combination of internal and external delivery threads.

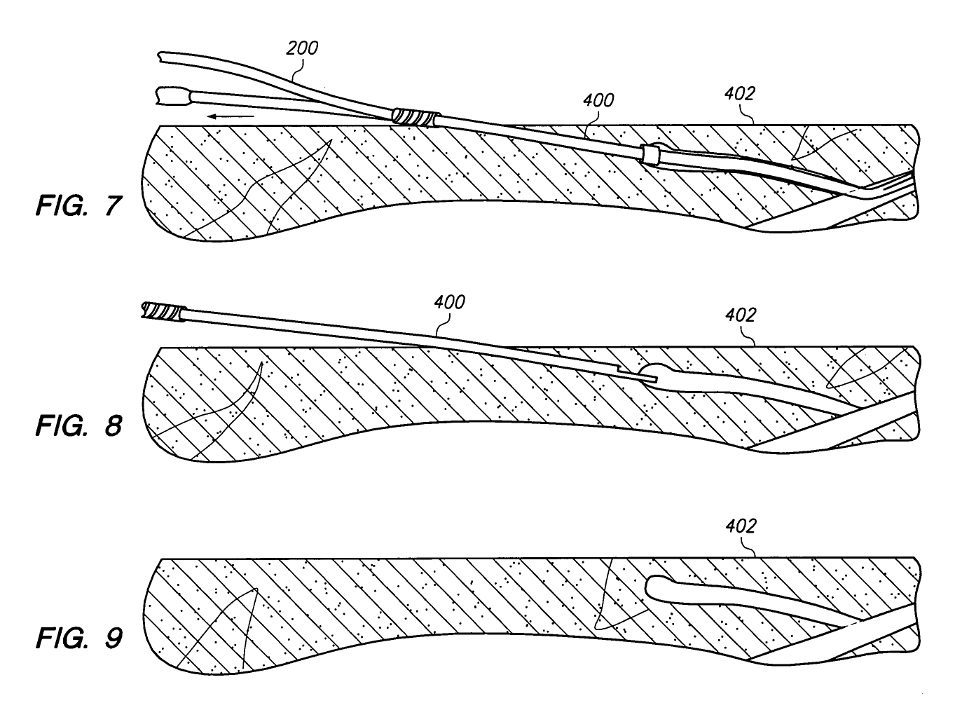

Reference is now made to FIGS. 4 through 9 individually and in combination for illustrating the insertion of the catheter removal device and removal of a catheter. Shown in FIG. 4 is a catheter 400 that is surgically implanted within a patient 402 and a catheter removal device 200 that is used to remove the catheter. In FIG. 5, the catheter removal device 200 is attached to the catheter 400 and begins advancing down the catheter towards a bacteriostatic cuff 500. The catheter removal device 200 is advanced by turning and applying a forward pressure on the device which results in the leading edge of the cutting tip separating/excising the tissue surrounding the catheter see FIG. 6. The catheter removal device 200 is continually twisted and pushed into the patient until the cutting tip of the device reaches the bacteriostatic cuff 500 at which point the cutting edge of the tip will excise the scar tissue surrounding the cuff. Once the scar tissue has been separated from the catheter and cuff area, the catheter has been liberated from the patient's tissue and subsequently the catheter removal device and the catheter may then be extracted in unison from the patient, See FIGS. 7 through 9.

The catheter removal device herein has several advantages over the current method of catheter removal. First, the new catheter removal device reduces the risk of medical complications because the cutting surfaces cleanly separate the tissue surrounding the catheter instead of tearing the tissues as currently practiced. As a result of using honed cutting surfaces, there is less tissue damage/trauma associated with the catheter removal process. Current methods and devices used for catheter removal employ blunt dissection in which there is typically extensive tissue damage/trauma because the tissues adhering to the catheter are torn and not severed with a sharp edged instrument.

Secondly, the new catheter removal device is optimized for quick and efficient removal of the catheter. Due to the efficiency of the catheter removal device, the procedure of catheter removal is much quicker and the patient will experience reduced procedural time and thus less stress regarding the procedure.

Thirdly, the new catheter removal device is inexpensive and can be manufactured as a disposable device. In contrast, the current method of removing a catheter uses a hemostat for blunt dissection which requires sterilization and inventory procedures.

Finally, another advantage is that the new catheter removal device and method of use are very intuitive and require minimal training. As a result, the new catheter removal device enables other medical professional to practice the procedure in a cost and time effective manner.

While various embodiments of the invention have been described, it will be apparent to those of ordinary skill in the art that many more embodiments and implementations are possible that are within the scope of this invention. In addition, the various features, elements, and embodiments described herein may be claimed or combined in any configuration or arrangement.

What is claimed is:

1. A catheter removal device comprising:
a handle;
a shaft having a distal end, the shaft extending from the handle;
a shaft longitudinal groove in the shaft extending through the distal end of the shaft; and
a cutting tip at the distal end of the shaft, the cutting tip comprising a cutting tip longitudinal groove, a plurality of delivery threads, a leading edge having a honed cutting surface; and an internal cutting edge formed on the cutting tip longitudinal groove.

2. The catheter removal device of claim 1, wherein the delivery threads are formed on the cutting tip longitudinal groove.

3. The catheter removal device of claim 1, wherein the delivery threads are formed on an exterior surface of the cutting tip.

4. The catheter removal device of claim 1, wherein the shaft longitudinal groove and the cutting tip longitudinal groove are substantially circular and sized to accommodate the outer dimension of a catheter tubing.

5. The catheter removal device of claim 1 wherein the shaft is semi-rigid.

6. A catheter removal device comprising:
a handle;
a shaft having a distal end, the shaft extending from the handle;
a shaft longitudinal groove in the shaft extending through the distal end of the shaft;
a cutting tip extending from the distal end of the shaft, the cutting tip comprising a cutting tip longitudinal groove and a leading edge having a honed cutting surface; and
a retractable cutting edge, the retractable cutting edge not extending into the cutting tip longitudinal groove when retracted and extending into the cutting tip longitudinal groove when deployed.

7. The catheter removal device of claim 6 further comprising:
a cutting switch, the cutting switch being movable from a first position to retract the cutting edge and to a second position to deploy the cutting edge;
a longitudinal cavity in the shaft; and
an activation rod, the activation rod having the cutting edge associated therewith and being operatively coupled with the cutting switch and the longitudinal cavity to allow the activation rod to longitudinally translate within the longitudinal cavity when the cutting switch is moved.

8. The catheter removal device of claim 7, wherein the cutting edge is integrally formed into the distal end of the activation rod.

9. The catheter removal device of claim 6, wherein the cutting tip further comprises a plurality of delivery threads.

10. The catheter removal device of claim 9, wherein the delivery threads are formed on the cutting tip longitudinal groove, on an exterior surface of the cutting tip, or both.

11. The catheter removal device of claim 6, wherein the shaft longitudinal groove and the cutting tip longitudinal groove are substantially circular and sized to accommodate the outer dimension of a catheter tubing.

12. The catheter removal device of claim 6, wherein the shaft is semi-rigid.

13. A method for removing a catheter comprising:
attaching a catheter removal device to an external portion of a catheter, the catheter removal device comprising a handle, a shaft, a cutting tip, and a longitudinal groove extending through the cutting tip to at least a portion of the shaft;
advancing the catheter removal device along the catheter until the cutting tip engages a cuff portion of the catheter;
separating tissue from around the catheter, the cuff portion, or both, wherein tissue is separated by applying both a turning force and a forward force to the handle of the catheter removal device; and
removing the catheter removal device and catheter upon successful separation of tissue from around the catheter, the cuff portion, or both.

14. The method of claim 13, wherein attaching a catheter removal device further comprises placing at least a portion of the longitudinal groove around an external portion of the catheter.

15. The method of claim 13, wherein the catheter removal device further comprises one or more delivery threads formed on the longitudinal groove within the cutting tip or on an exterior surface of the cutting tip.

16. The method of claim 15, wherein advancing the catheter removal device further comprises advancing the catheter removal device into a patient along the catheter by applying a turning force to the one or more delivery threads through rotating the handle of the catheter removal device.

17. The method of claim 13, wherein the catheter removal device further comprises a cutting switch and an activation rod, the activation rod operatively connected to the cutting edge at a distal end and the cutting switch at an opposite end.

18. The method of claim 17 further comprising the steps of:
deploying the cutting edge to separate tissue from around the catheter, the cuff portion, or both, wherein the cutting edge is deployed by moving the cutting switch to a first position; and
retracting the cutting edge prior to removing the catheter removal device and the catheter, wherein the cutting edge is retracted by moving the cutting switch to a second position.
